(19) United States
(12) Patent Application Publication
(10) Pub. No.: US 2026/0006983 A1
(43) Pub. Date: Jan. 1, 2026

(54) LIGHT EMITTING DEVICE COMPRISING OPTICAL GUIDE ELEMENT

(71) Applicant: Commissariat à I'Énergie Atomique et aux Énergies Alternatives, Paris (FR)

(72) Inventors: Benoît Racine, Grenoble (FR); Guillaume Blanquer, Grenoble (FR); Anne Planat-Chretien, Grenoble (FR); Christelle Laugier, Grenoble (FR)

(73) Assignee: Commissariat à I'Énergie Atomique et aux Énergies Alternatives, Paris (FR)

(21) Appl. No.: 19/237,396

(22) Filed: Jun. 13, 2025

(30) Foreign Application Priority Data
Jun. 17, 2024 (FR) FR2406386

(51) Int. Cl. H10K 50/858; H10K 50/856

(52) U.S. Cl. CPC H10K 50/858; H10K 50/856

(57) ABSTRACT

A light-emitting device including at least: an optical guide element; a light source at least partially transparent to at least one type of light intended to be emitted by the light source, and configured to emit the light at least on the side of a first surface arranged in front of the optical guide element and on the side of a second surface opposite to the first surface; a reflective layer arranged on the side of the second surface of the light source; an optical component arranged between the reflective layer and the optical guide element and at least partially transparent to the light intended to be emitted by the light source; and wherein the reflective layer forms at least one reflective surface conformal to a non-planar surface of the optical component having the reflective layer arranged thereon.

### CROSS REFERENCE TO RELATED APPLICATIONS

This application claims priority to French application number FR2406386, filed Jun. 17, 2024. The contents of this application are incorporated by reference in its entirety.

### TECHNICAL FIELD

The present disclosure generally concerns light-emitting devices with an optical guide element, or light guide element.

### PRIOR ART

Photodynamic therapy, or PDT, is a treatment technique that destroys tissue of tumoral or non-tumoral origin through the joint action of a photosensitive active principle injected into the tissue and of an illumination of the tissue by a light source at an appropriate wavelength (in the visible range) to enable photoactivation of the active principle, leading to destruction of the targeted tissue through cell death. The reaction that takes place is complex and involves the local presence of oxygen, and thus the presence of a regional perfusion. One of the main problems with this technique is the short distance of penetration of light into the tissue. Indeed, light does not propagate sufficiently inside the skin due to the absorption and to the scattering of photons in the tissue to be crossed, and thus cannot properly activate the photosensitive molecules injected into the tissue, which are distributed from the surface up to several hundred micrometers below the skin surface, depending on the injection mode used. As a result, the efficacy of the treatment is greatly reduced, and only pathologies close to the skin surface can be treated by this technique.

To overcome this problem, it is possible to use a very high light intensity and/or longer exposure times (within the limits of regulatory standards) to provide the quantity of optical energy needed for the activation of the photosensitive molecules inside the tissue. But in this case, it is possible to damage the microcirculation of neighboring healthy tissue, or the microcirculation of overexposed superficial layers, or even to fail to achieve treatment efficacy at the desired depth. In addition, under certain lighting conditions, this exposure may cause severe pain (strong photochemical burns).

Another solution to the problem of light scattering in tissue consists in using microneedles forming light guides. These microneedles are inserted into the skin and tissue down to a depth slightly smaller than the length of the microneedle, for example several hundred microns. Due to this configuration, it is possible to irradiate the deep layer of tissue via the light guiding performed within the microneedles, and thus decrease the skin irradiation dose at the surface, while efficiently reaching deep areas. Light can be introduced into the microneedles by wide-field illumination or via microlenses arranged above the microneedles. The use of microlenses enables light to be focused directly into the microneedles. In this solution, the light is thus directed towards the microneedles for a better distribution of light in the skin. However, the surface illumination is no longer performed, or much less efficiently.

Similar optical guiding problems can be encountered in other fields such as that of optical communications, for example when the coupling of light in a waveguide needs to be optimized.

### SUMMARY OF THE INVENTION

There is a need to provide a solution overcoming at least some of the above-discussed disadvantages.

An embodiment overcomes all or part of these disadvantages and provides a light-emitting device comprising at least:
an optical guide element;
a light source at least partially transparent to at least one type of light to be emitted by the light source, and configured to emit the light at least on the side of a first surface arranged in front of the optical guide element and on the side of a second surface opposite to the first surface;
a reflective layer on the side of the second surface of the light source;
an optical component arranged between the reflective layer and the optical guide element and at least partially transparent to the light intended to be emitted by the light source;
and wherein the reflective layer forms at least one reflective surface conformal to a non-planar surface of the optical component having the reflective layer arranged thereon.

According to a specific embodiment, the light source comprises at least one organic light-emitting diode.

According to a specific embodiment, the reflective layer is one of the electrodes of the organic light-emitting diode.

According to a specific embodiment, the reflective surface comprises at least one concave or convex portion.

According to a specific embodiment, the reflective surface forms at least one spherical, or conical, or hyperbolic mirror.

According to a specific embodiment, the reflective layer comprises at least one metal layer and/or at least one Bragg mirror.

According to a specific embodiment, the light-emitting device further comprises at least one substrate at least partially transparent to the light intended to be emitted by the light source and arranged between the optical guide element and the light source.

According to a specific embodiment, the optical guide element comprises at least one microneedle at least partially transparent to the light intended to be emitted by the light source, or at least one waveguide.

According to a specific embodiment, the optical guide element comprises a base at least partially transparent to the light intended to be emitted by the light source, and a plurality of microneedles at least partially transparent to the light intended to be emitted by the light source and each comprising a first end integral with the base, the base being arranged between the light source and the microneedles.

According to a specific embodiment, the optical guide element comprises a plurality of microneedles at least partially transparent to the light intended to be emitted by the light source, and wherein the optical component comprises a layer of material at least partially transparent to the light intended to be emitted by the light source, the layer of material comprising recesses aligned with the microneedles.

According to a specific embodiment, the light source comprises a plurality of distinct parts configured to emit light of different wavelengths, each of said parts being arranged in front of at least one of the microneedles.

According to a specific embodiment, the light source is configured to emit part of the light between the microneedles.

According to a specific embodiment, at least part of the reflective surface is configured to reflect part of the light emitted on the side of the second surface of the light source between the microneedles.

There is also provided a method of manufacturing a light-emitting device, comprising at least:
the forming of at least one optical guide element;
the forming of at least one light source at least partially transparent to at least one type of light intended to be emitted by the light source, and configured to emit the light at least on the side of a first surface arranged in front of the optical guide element and on the side of a second surface opposite to the first surface;
the forming of at least one reflective layer arranged on the side of the second surface of the light source;
the forming of at least one optical component arranged between the reflective layer and the optical guide element and at least partially transparent to the light intended to be emitted by the light source;
and wherein the reflective layer is formed in such a way that it forms at least one reflective surface conformal to a non-planar surface of the optical component having the reflective layer arranged thereon.

According to a specific embodiment:
the light source and the reflective layer are formed on the optical guide element, or
the light source and the reflective layer are formed on a substrate at least partially transparent to the light intended to be emitted by the light source, the substrate then being bonded to the optical guide element, or
the light source, the reflective layer, and the optical component are formed on a substrate opaque to the light intended to be emitted by the light source, the optical component then being bonded to the optical guide element.

### BRIEF DESCRIPTION OF THE DRAWINGS

The foregoing features and advantages, as well as others, will be described in detail in the rest of the disclosure of specific embodiments given as an illustration and not limitation with reference to the accompanying drawings, in which:

FIG. 1 schematically shows an example of a light-emitting device according to a first embodiment;

FIG. 2 shows examples of focusing of light obtained with different reflective surface shapes in a light-emitting device;

FIG. 3, FIG. 4, FIG. 5, FIG. 6, FIG. 7, FIG. 8, FIG. 9, and FIG. 10 show steps of an example of a method of forming a light-emitting device according to the first embodiment;

FIG. 11 schematically shows an example of a light-emitting device according to a variant of the first embodiment;

FIG. 12 schematically shows an example of a light-emitting device according to a second embodiment;

FIG. 13 schematically shows an example of a light-emitting device according to a variant of the second embodiment;

FIG. 14, FIG. 15, FIG. 16, and FIG. 17 show steps of a method of forming a light-emitting device according to the second embodiment;

FIG. 18, FIG. 19, FIG. 20, and FIG. 21 show steps of a method of forming a light-emitting device according to another variant of the second embodiment.

### DESCRIPTION OF EMBODIMENTS

Like features have been designated by like references in the various figures. In particular, the structural and/or functional features that are common among the various embodiments may have the same references and may dispose identical structural, dimensional and material properties.

In the drawings, to make their reading easier, the various elements and the various layers of materials are not shown to the same scale with respect to one another.

For clarity, only those steps and elements which are useful to the understanding of the described embodiments have been shown and are described in detail.

Unless indicated otherwise, when reference is made to two elements connected together, this signifies a direct connection without any intermediate elements other than conductors, and when reference is made to two elements coupled together, this signifies that these two elements can be connected or they can be coupled via one or more other elements.

In the following description, where reference is made to absolute position qualifiers, such as the terms “front”, “back”, “top”, “bottom”, “left”, “right”, etc., or relative position qualifiers, such as the terms “top”, “bottom”, “upper”, “lower”, etc., or orientation qualifiers, such as “horizontal”, “vertical”, etc., reference is made unless otherwise specified to the orientation of the drawings. However, these terms do not presume the real position and orientation of the device during its use.

Unless specified otherwise, the expressions “about”, “approximately”, “substantially”, and “in the order of” signify plus or minus 10%, preferably of plus or minus 5%.

Throughout the document, the expression “at least partially transparent” is used to characterize the fact that an element can be crossed by at least part (for example at least 40% or at least 50% or at least 70% or at least 90%) of the light received at the input of this element and/or emitted by this element.

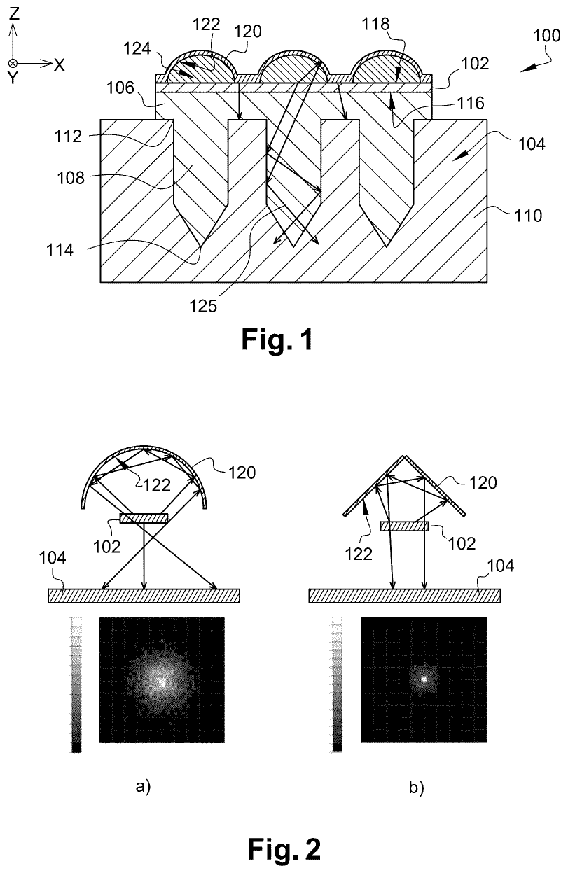

An example of a light-emitting device 100 according to a first embodiment is described hereafter in relation with FIG. 1. In this example of embodiment, device 100 corresponds to a photodynamic therapy device intended to emit light both at the surface of skin tissue and also deep into the tissue, below the external surface of the skin.

Device 100 comprises at least one light source 102 at least partially transparent (and preferably transparent) to at least one type of light intended to be emitted by light source 102. In the described embodiment, light source 102 is organic in nature and comprises an organic light-emitting diode, or OLED. This OLED comprises first and second electrodes (one corresponding to the anode of the OLED and the other corresponding to the cathode of the OLED) based on at least one electrically-conductive material, and at least one emissive layer based on at least one organic semiconductor material and arranged between the first and second electrodes. The thickness (dimension parallel to the Z axis in FIG. 1) of the OLED is, for example, in the range from 50 nm to 500 nm. As an example, the electrodes of the OLED may comprise at least one of the following materials: Al, Ag, ITO, SnO₂, etc. The emissive layer of the OLED may comprise, according to the desired wavelength, at least one of the following types of materials: Irppy, TADF (“thermally activated delayed fluorescent”), MR-TADF (“multiple resonance thermally activated delayed fluorescent”). Further, light source 102 is configured to emit light from at least two opposite surfaces.

A light source 102 comprising at least one OLED used in device 100 has the advantage, over other light source types, of achieving a homogeneous, isotropic light emission over a large surface area. As a variant, device 100 may comprise other types of light source 102 configured to emit from two opposite sides and which is at least partially transparent to the emitted light.

Device 100 further comprises at least one optical guide element 104. In the described embodiment, optical guide element 104 comprises a base 106 at least partially transparent (and preferably transparent) to the light intended to be emitted by light source 102, as well as a plurality of microneedles 108 also at least partially transparent (and preferably transparent) to the light intended to be emitted by light source 102 and intended to be inserted into tissue 110 corresponding to superficial layers of the skin. Each of microneedles 108 comprises a first end 112 integral with base 106 and a point-shaped second end 114. More particularly, in the described example, each microneedle 108 comprises a cylindrical portion extending from the first end 112 and continuing in a tapered portion all the way to the second end 114. As a variant, shapes of microneedle 108 other than that described hereabove are possible. For example, the cross-section of microneedles 108 may be of a shape other than a disk. Further, shapes other than a point are conceivable, for example to orient optical guide element 104 or to couple it with a local diffuser arranged at the tip of microneedles 108.

In the described example of embodiment, microneedles 108 enable to guide the light received at the surface of the base 106 having light source 102 arranged thereon, into the deeper layers of the skin having microneedles 108 inserted therein. The conical shape of the tips of microneedles 108 enables to drive microneedles 108 well into the skin, and also to ensure a good scattering of the light guided in the cylindrical portion of microneedles 108. As an example, the height of each of microneedles 108 (dimension parallel to the Z axis in FIG. 1) may be in the range from 100 μm to 3 mm. The cross-section of the cylindrical portion of each of microneedles 108, in a plane perpendicular to their height (plane parallel to the (X,Y) plane in the example of FIG. 1) has, for example, a diameter in the range from 50 μm to 900 μm. The pitch of microneedles 108, that is, the distance separating the axes of revolution of two neighboring microneedles 108, may be in the range from approximately 100 μm to several millimeters. According to an embodiment, microneedles 108 may comprise a biocompatible material such as polymethyl methacrylate or PMMA, or PLGA (poly (lactic-co-glycolic acid)).

When device 100 is intended for uses other than photodynamic therapy, optical guide element 104 may comprise base 106 to which are optically coupled one or more elements at least partially transparent (and preferably transparent) to the light intended to be emitted by light source 102, which element(s) may be other than microneedles.

Thus, optical guide element 104 may comprise, for example, at least one microneedle, or at least one waveguide (example of an application other than photodynamic therapy), and optical guide element 104 may or may not comprise base 106.

Light source 102 is configured to emit light at least on the side of a first surface 116 arranged in front of optical guide element 104 and also on the side of a second surface 118 opposite to first surface 116. This light emission on the side of each of surfaces 116, 118 is due, in this example of embodiment, to the fact that light source 102 is an OLED which emits light on the side of each of its electrodes (first electrode arranged on side of first surface 116 and second electrode arranged on the side of second surface 118). As a variant, this light emission from both surfaces 116, 118 of light source 102 can be obtained using other types of light source 102.

In the described example of embodiment, the first surface 116 of light source 102 is arranged directly against optical guide element 104, and more particularly against the base 106 of optical guide element 104. As a variant, it is possible for at least one element at least partially transparent, or preferably transparent, to the light intended to be emitted by light source 102, for example a substrate of glass or any other transparent or semi-transparent material, to be interposed between light source 102 and optical guide element 104, such as for example conical bases having the microneedles arranged thereon, and with a possible baseplate having the conical bases resting thereon.

Device 100 further comprises at least one reflective layer 120 arranged on the side of the second surface 118 of light source 102 and forming a reflective surface 122 on the side of light source 102. This surface 122 is said to be reflective due to the fact that it is configured to reflect at least part of, and preferably all or almost all, the light emitted by light source 102 on the side of its second surface 118. Reflective layer 120 comprises, for example, at least one metal such as silver or aluminum. The thickness of reflective layer 120 is for example in the range from 50 nm to 500 nm.

As a variant, reflective layer 120 may comprise at least one Bragg mirror configured to reflect the wavelength(s) of interest emitted by light source 102 on the side of its second surface 118, that is, the light intended to be sent towards tissue 110.

In any case, the properties of reflective layer 120 (material(s) used, thickness, shape, etc.) may be such that reflective surface 122 reflects as much light as possible in order to have the lowest possible light loss in this reflective layer 120.

Device 100 further comprises at least one optical component 124 arranged between reflective layer 120 and optical guide element 104, and more particularly between light source 102 and reflective layer 120 in the first embodiment. In the example of FIG. 1, device 100 comprises a plurality of optical components 124, each arranged in front of one of microneedles 108. The pitch (distance between the centers of two neighboring optical components 124) with which optical components 124 are formed may be equal to that of microneedles 108. Optical components 124 are at least partially transparent, and preferably transparent, to the light intended to be emitted by light source 102 on the side of its second surface 118. According to an example of embodiment, optical components 124 comprise a resin-type polymer or an oxide such as SiO₂ or SiN or any other suitable material. Further, the thickness of each of optical components 124 (that is, their dimension parallel to the Z axis in the example of FIG. 1) is for example in the range from 50 μm to 2 mm.

Reflective layer 120 is arranged on the optical components 124 in such a way that reflective surface 122 is conformal to a non-planar surface of optical components 124 and thus achieves a light reflection according to a desired directivity and/or focus. Thus, the geometry of the reflective surface 122 facing each microneedle 108 depends on that of the non-planar surface of each optical component 124. In the described example of embodiment, each optical component 124 forms a concave surface having reflective layer 120 arranged thereon, this shape corresponding to that of reflective surface 122. For example, optical components 124 may be such that reflective surface 122 forms, in front of each microneedle 108, at least one spherical mirror, or spherical cap, which may also be conical or hyperbolic. As a variant, each optical component 124 may have a convex or other non-planar shape adapted to achieving a desired light reflection. For example, optical components 124 may be such that, when combined with reflective surface 122, they enable to locally increase the directivity of light to reflect it with a suitable angle into optical guide element 104.

Further, optical components 124 combined with reflective surface 122 may be configured to focus the light emitted by light source 102 on the side of the second surface 118 in each of microneedles 108, as illustrated by the arrows designated with reference numeral 125. The selection of the shape of the non-planar surface of optical components 124 having the reflective surface 122 of reflective layer 120 arranged thereon may depend on the desired light focus in optical guide element 104, and more particularly in microneedles 108 in the example described example. In FIG. 2, view a) shows the focus obtained on the surface of an optical guide element 104 having light entering therethrough, when reflective surface 122 forms a spherical mirror, and view b) represents the focus obtained when reflective surface 122 forms a conical mirror. These drawings show that the achieved focus is greater when reflective surface 122 forms a conical mirror than when it forms a spherical mirror.

In the first embodiment, light source 102 is configured to emit, from its first surface 116, part of the light into microneedles 108 and another part of the light between microneedles 108. Light source 102 is also configured to emit, from its second surface 118, part of the light into microneedles 108 after it has crossed optical components 124, reflected off reflective surface 122, and crossed again optical components 124 and light source 102, and another part of the light between microneedles 108 after it has reflected off the portions of reflective layer 120 arranged between optical components 124 and crossed light source 102. Indeed, the portions of the second surface 118 of light source 102 not covered by optical components 124 are directly covered by reflective layer 120. Thus, in the described example of embodiment, this part of the light sent between microneedles 108 directly enters tissue 110 from the external surface of the skin, while the part of the light sent into microneedles 108 reflects against the walls of the cylindrical portion of microneedles 108 before coming out into tissue 110 in the conical portions of microneedles 108. Thus, due to device 100, light is sent both to the surface of tissue 110 (corresponding to the surface irradiation of the skin) and also to different depths in tissue 110. The light sent into tissue 110 by device 100 is thus not concentrated only at the surface or only deep within tissue 110. This enables, in the case of a use of device 100 for photodynamic therapy, to obtain an optimum efficacy of the treatment performed, at different depths in tissue 110.

As variant of the above-described first embodiment, optical guide element 104 may correspond to a waveguide. In this case, reflective layer 120 and optical component(s) 124 enable to increase the light intensity sent into this waveguide, due to the fact that the light emitted from the second surface 118 of light source 102 can be reflected and focused towards the waveguide. The device 100 according to such a variant may for example be used in the field of optical communications in order to optimize the coupling of light in the waveguide corresponding to optical guide element 104.

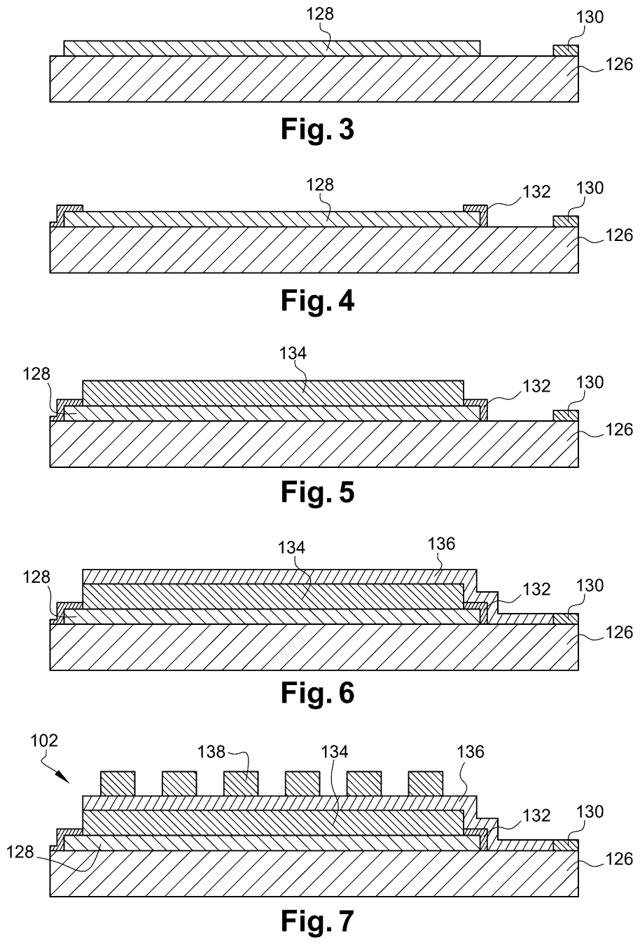

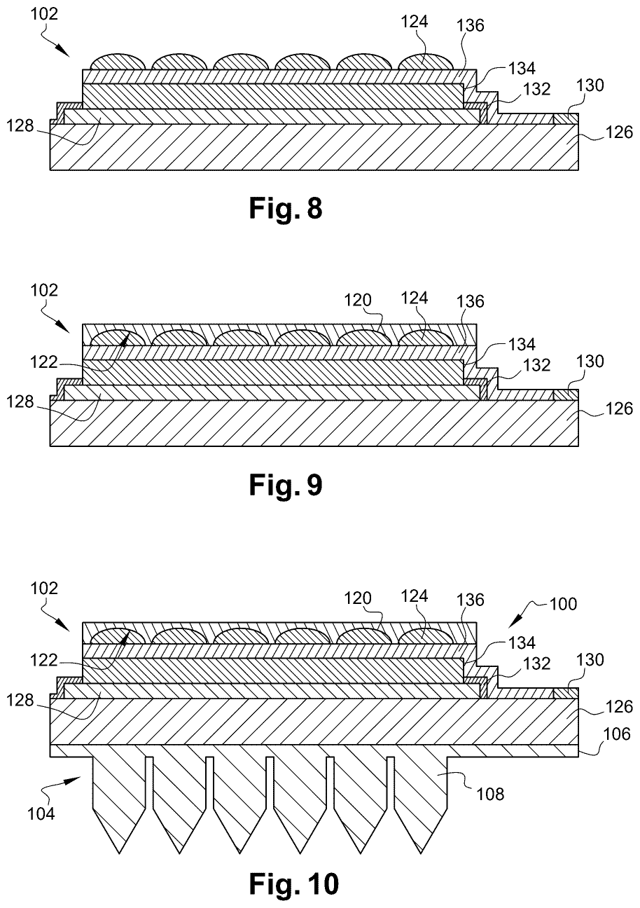

An example of a method of forming the device 100 according to the first embodiment is described hereafter in relation with FIGS. 3 to 10.

In this example, light source 102, reflective layer 120, and optical components 124 are formed on a substrate 126 at least partially transparent to the light intended to be emitted by light source 102. The thickness of substrate 126 is, for example, equal to a few hundred microns. As a variant, it is possible for light source 102, reflective layer 120, and optical components 124 to be formed directly on optical guide element 104, as is the case in the example of FIG. 1.

In the described example of embodiment, the obtained light source 102 corresponds to an OLED. Thus, in this example, a first electrode 128, transparent or semi-transparent, that is, capable of letting through at least part of the light intended to be emitted from the emissive layer(s) of light source 102, is formed on substrate 126. First electrode 128 corresponds, for example, to the anode of the OLED forming light source 102. A contact pad 130 to which a second electrode of light source 102 is intended to be electrically coupled is also formed on substrate 126, next to the first electrode 128 (see FIG. 3).

An insulating portion 132, for example comprising resin, is then formed, for example by deposition, at the periphery of first electrode 128 (see FIG. 4). This insulating portion 132 is designed to electrically insulate first electrode 128 from the electrical connection that will be made between the second electrode of light source 102 and contact pad 130.

One or more emissive layers 134, here comprising at least one organic material, are then deposited on first electrode 128 (see FIG. 5).

A second transparent or semi-transparent electrode 136 is formed on emissive layer(s) 134. This second electrode 136 corresponds, for example, to the cathode of the OLED forming light source 102. Part of this second electrode 136 is deposited on at least one sidewall of emissive layer(s) 134, on part of insulating portion 132, and on part of substrate 126 so as to be in contact with contact pad 130 (see FIG. 6). At this stage of the method, the forming of light source 102 has been completed.

Although not shown, a transparent or semi-transparent encapsulation layer may be deposited on light source 102.

Optical components 124 are then formed on light source 102. In the described example of embodiment, pads 138 of the material intended to form optical components 124, for example transparent or semi-transparent resin pads, are formed for example by deposition above the second electrode 136, for example on the encapsulation layer (see FIG. 7).

A creep step may then be implemented to give pads 138 the desired shape and thus form optical components 124 (see FIG. 8).

Reflective layer 120 is then formed, for example by deposition, on optical components 124 and on the portions of second electrode 136 not covered by optical components 124 (see FIG. 9).

Device 100 is completed by transferring the resulting structure onto guide element 104, comprising, in the described example of embodiment, base 106 and microneedles 108. This transfer corresponds, in the described example, to a bonding of substrate 126 to base 106 (see FIG. 10).

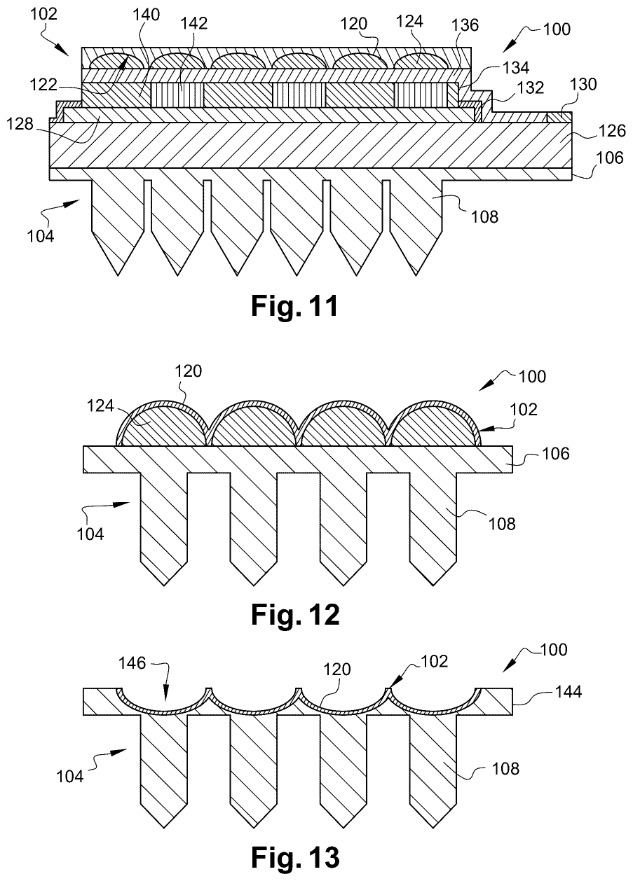

In a variant, light source 102 may be formed in such a way that it comprises a plurality of distinct parts configured to emit light of different wavelengths, and which are each arranged in front of at least one of microneedles 108. An example of embodiment of a device 100 according to such a variant is shown in FIG. 11. In this drawing, emissive layer(s) 134 comprise first emissive portions 140 and second emissive portions 142 arranged in alternated fashion next to one another on first electrode 128. According to an example of embodiment, the first emissive parts 140 may be configured to emit red light (which has the property of well penetrating the epidermis), and the second emissive parts 142 may be configured to emit blue light (which has the property of being well absorbed by the active principle used in phototherapy treatments).

As a variant, light source 102 may be configured to emit wavelengths different from the examples described hereabove, and/or a greater number of different wavelengths.

An example of a light-emitting device 100 according to a second embodiment is described hereafter in relation with FIG. 12.

In this second embodiment, device 100 comprises optical guide element 104 formed of base 106 and of microneedles 108. Optical components 124 are arranged on the base 106 of optical guide element 104.

Conversely to the first embodiment, in which light source 102 is arranged between optical guide element 104 and optical components 124, and with reflective layer 120 formed above optical components 124, optical components 124 are here arranged on optical guide element 104, with light source 102 arranged on optical components 124. In other words, optical components 124 are here arranged between optical guide element 104 and light source 102. Further, in this second embodiment, reflective layer 120 corresponds to one of the OLED electrodes forming light source 102 and corresponds to that forming an external layer of light source 102 (that is, that which is not arranged directly against optical components 124). For this electrode to form reflective layer 120, and thus reflective surface 122, this electrode comprises, for example, at least one of the following materials: Ag, Al, Au, Cr, etc., as well as a thickness sufficient for this layer to be opaque and reflective.

In this second embodiment, the various layers of light source 102 (electrodes and emissive layer(s)) are conformally deposited on the non-planar surfaces formed by optical components 124. As compared with a planar light source 102 as previously described in relation with the first embodiment, the emissive surface area of the light source 102 according to the second embodiment is larger, for a given footprint on the surface of optical guide element 104, which enables to increase the amount of light sent into optical guide element 104. In the example of FIG. 12, by forming optical components 124 in such a way that they are arranged next to each other and touching, the amount of light emitted may be approximately six times greater than in the case of a planar light source 102.

In the example of embodiment shown in FIG. 12, optical components 124 have a concave shape, which means that the reflective surface formed by reflective layer 120 is also concave.

In a variant shown in FIG. 13, the optical components 124 of device 100 are formed from a layer of material 144 at least partially transparent to the light to be emitted by light source 102, this layer of material 144 comprising recesses 146 arranged vertically in line with microneedles 108. Thus, these recesses 146 form convex surfaces such that the reflective surfaces formed by reflective layer 120 in front of microneedles 108 are also convex. Further, in this variant, optical guide element 104 does not comprise base 106, but only microneedles 108. As in the previously described example, this variant enables to obtain a larger emissive surface area of light source 102, for a given footprint on the surface of optical guide element 104, and thus to increase the amount of light sent into optical guide element 104.

The different variants previously described for the first embodiment may apply to this second embodiment.

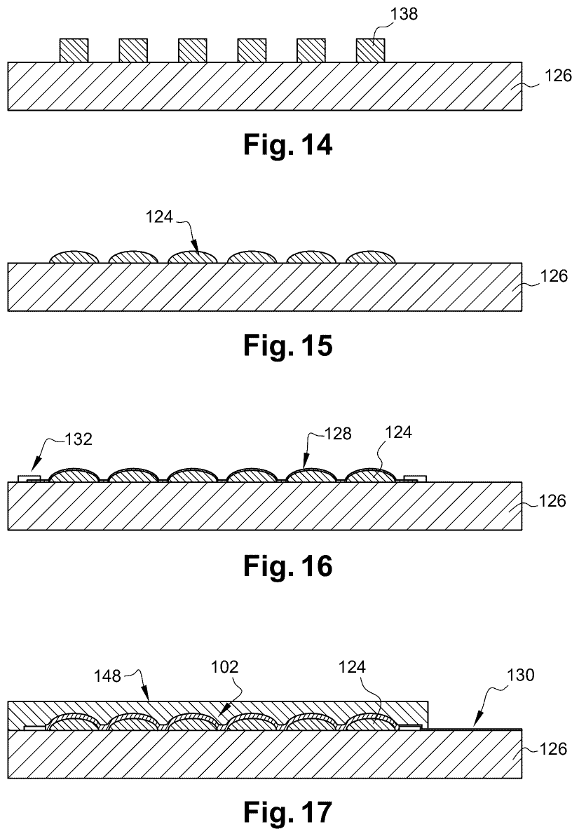

An example of a method of forming the device 100 according to the second embodiment is described hereafter in relation with FIGS. 14 to 17.

In this example, optical components 124 are first formed by depositing on substrate 126 at least one layer of transparent or semi-transparent material intended for the forming of optical components 124. This layer of material comprises, for example, transparent or semi-transparent resin. Photolithography and development steps may then be implemented to form the pads 138 of material, for example, similar to those previously described for the first embodiment. The structure obtained at this stage of the method is shown in FIG. 14.

A creep step may then be implemented to form optical components 124 (see FIG. 15).

First electrode 128 is then formed, for example by deposition, on optical components 124 as well as on portions of substrate 126, between and next to optical components 124. As in the first embodiment, insulating portion 132 is then formed (see FIG. 16).

Light source 102 is then completed by depositing emissive layer(s) 134, and then second electrode 136 (with, in the described example, the forming of contact pad 130). The assembly is then covered with an encapsulation layer 148, which may be transparent or opaque (see FIG. 17).

Device 100 is then completed by transferring substrate 126 onto an optical guide element 104, for example similar to one of the previously-described examples.

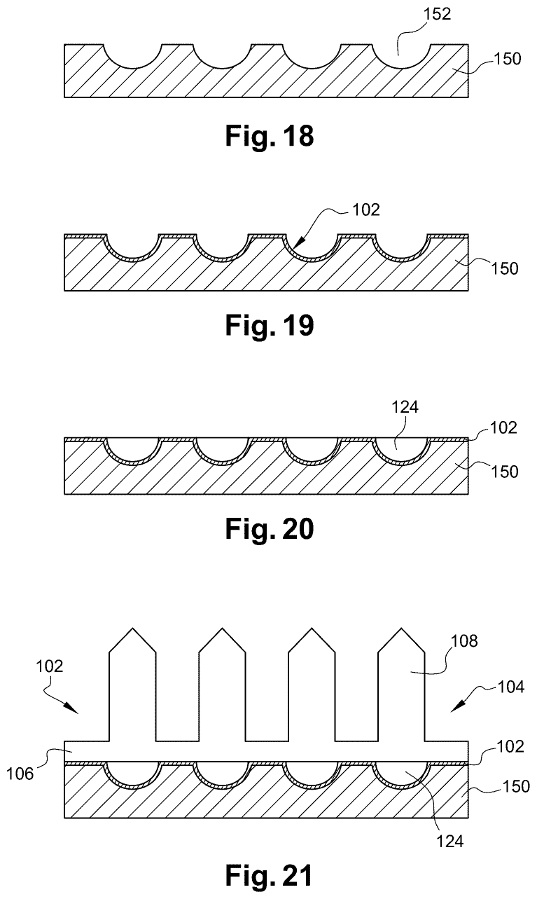

According to another embodiment described in relation with FIGS. 18 to 21, device 100 may be formed from an opaque substrate 150.

Opaque substrate 150 is first etched on one of its surfaces, forming recesses 152 forming convex surfaces (see FIG. 18). A first electrode is then deposited onto the previously etched surface of substrate 150, and also in recesses 152, so as to form the anode and the reflective surface. One or more emissive layers are then deposited on the first electrode. A second electrode is then deposited on the emissive layer(s), completing the forming of light source 102. A transparent or semi-transparent encapsulation layer is eventually deposited on the second electrode (see FIG. 19). The remaining volume of recesses 152 which is not occupied by the layers deposited to form light source 102 and by the encapsulation layer is filled with a material forming optical components 124 (see FIG. 20). The resulting assembly is then transferred, for example by bonding, to a substrate comprising optical guide element(s) 104 (see FIG. 21).

In all embodiments, device 100 enables to optimize the injection of light into optical guide element 104 due to the judicious use of non-planar reflective surface 122, and which, when combined with a light source 102 emitting light both on the side of optical guide element 104 and on the side of reflective surface 102, enables to increase the amount of light sent to optical guide element 104, given that the light emitted on the side of reflecting surface 122 is recovered and reflected towards optical guide element 104 due to the light-reflecting and light-focusing properties of reflective surface 122.

Various embodiments and variants have been described. Those skilled in the art will understand that certain features of these various embodiments and variants may be combined, and other variants will occur to those skilled in the art.

Finally, the practical implementation of the described embodiments and variants is within the abilities of those skilled in the art based on the functional indications given hereabove. For example, the precise nature of the implemented deposition and etch steps may be selected in particular as a function of the material(s) to be deposited or to be etched, as well as of the thicknesses of the materials to be deposited or to be etched.

1. Light-emitting device comprising at least:
an optical guide element;
a light source at least partially transparent to at least one type of light intended to be emitted by the light source, and configured to emit the light at least on the side of a first surface arranged in front of the optical guide element and on the side of a second surface opposite to the first surface;
a reflective layer arranged on the side of the second surface of the light source;
an optical component arranged between the reflective layer and the optical guide element and at least partially transparent to the light intended to be emitted by the light source;
and wherein the reflective layer forms at least one reflective surface conformal to a non-planar surface of the optical component having the reflective layer arranged thereon.

2. Light-emitting device according to claim 1, wherein the light source comprises at least one organic light-emitting diode.

3. Light-emitting device according to claim 2, wherein the reflective layer is one of the electrodes of the organic light-emitting diode.

4. Light-emitting device according to claim 1, wherein the reflective surface comprises at least one concave or convex portion.

5. Light-emitting device according to claim 1, wherein the reflective surface forms at least one spherical or conical or hyperbolic mirror.

6. Light-emitting device according to claim 1, wherein the reflective layer comprises at least one metal layer and/or at least one Bragg mirror.

7. Light-emitting device according to claim 1, further comprising at least one substrate at least partially transparent to the light intended to be emitted by the light source and arranged between the optical guide element and the light source.

8. Light-emitting device according to claim 1, wherein the optical guide element comprises at least one microneedle at least partially transparent to the light intended to be emitted by the light source, or at least one waveguide.

9. Light-emitting device according to claim 8, wherein the optical guide element comprises a base at least partially transparent to the light intended to be emitted by the light source and a plurality of microneedles at least partially transparent to the light intended to be emitted by the light source and each comprising a first end integral with the base, the base being arranged between the light source and the microneedles.

10. Light-emitting device according to claim 8, wherein the optical guide element comprises a plurality of microneedles at least partially transparent to the light intended to be emitted by the light source, and wherein the optical component comprises a layer of material at least partially transparent to the light intended to be emitted by the light source, the layer of material comprising recesses aligned with the microneedles.

11. Light-emitting device according to claim 9, wherein the light source comprises a plurality of distinct parts configured to emit light of different wavelengths, each of said parts being arranged in front of at least one of the microneedles.

12. Light-emitting device according to claim 9, wherein the light source is configured to emit part of the light between the microneedles.

13. Light-emitting device according to claim 9, wherein at least part of the reflective surface is configured to reflect part of the light emitted on the side of the second surface of the light source between the microneedles.

14. Method of manufacturing a light-emitting device, comprising at least:
the forming of at least one optical guide element;
the forming of at least one light source at least partially transparent to at least one type of light intended to be emitted by the light source, and configured to emit the light at least on the side of a first surface arranged in front of the optical guide element and on the side of a second surface opposite to the first surface;
the forming of at least one reflective layer arranged on the side of the second surface of the light source;
the forming of at least one optical component arranged between the reflective layer and the optical guide element and at least partially transparent to the light intended to be emitted by the light source;
and wherein the reflective layer is designed in such a way that it forms at least one reflective surface conformal to a non-planar surface of the optical component having the reflective layer arranged thereon.

15. Manufacturing method according to claim 14, wherein:
the light source and the reflective layer are formed on the optical guide element, or
the light source and the reflective layer are formed on a substrate at least partially transparent to the light intended to be emitted by the light source, the substrate then being bonded to the optical guide element, or
the light source, the reflective layer, and the optical component are formed on a substrate opaque to the light intended to be emitted by the light source, the optical component then being bonded to the optical guide element.
